(19) United States
(12) Patent Application Publication
(10) Pub. No.: US 2013/0158666 A1
(43) Pub. Date: Jun. 20, 2013

(54) FACET FUSION SYSTEM

(71) Applicants: Robert L. Assell, St. Paul, MN (US); Brian P. Beaubien, St. Paul, MN (US); Corinne Thomford, St. Paul, MN (US)

(72) Inventors: Robert L. Assell, St. Paul, MN (US); Brian P. Beaubien, St. Paul, MN (US); Corinne Thomford, St. Paul, MN (US)

(73) Assignee: Zyga Technology, Inc., Minneapolis, MN (US)

(21) Appl. No.: 13/525,834

(22) Filed: Jun. 18, 2012

Related U.S. Application Data
(60) Provisional application No. 61/497,769, filed on Jun. 16, 2011.

(51) Int. Cl. A61F 2/44; A61B 17/17

(52) U.S. Cl. CPC A61F 2/4455; A61B 17/1757
USPC 623/17.16; 606/96

(57) ABSTRACT

A facet fusion system including a first drill guide, a second drill guide and a facet fusion implant. The first drill guide has a first aperture formed therein. The second drill guide has a second aperture formed therein. A position of the first aperture on the first drill guide is offset from a position of the second aperture on the second drill guide. The facet fusion implant has at least one gripping region and at least one bone growth region. The at least one bone growth region has a diameter that is larger than a diameter of the first aperture and the second aperture.

### REFERENCE TO RELATED

This application claims priority to U.S. Provisional Application No. 61/497,769, which was filed on Jun. 16, 2011, the contents of which are incorporated herein by reference.

### FIELD OF THE INVENTION

The invention relates generally to a system for treating the facet joint. More particularly, the invention relates to a system for use in performing fusion of a facet joint.

### BACKGROUND OF THE INVENTION

Skeletal structures are formed of bones and adjoining structures that include cartilage, for instance. The human spine serves many functions. The vertebral members of the spinal column protect the spinal cord. The spinal column also supports other portions of the human body. The human spine is composed of a column of thirty-three bones, called vertebrae, and their adjoining structures.

The twenty-four vertebrae nearest the head are separate bones capable of individual movement. These vertebrae are interconnected by anterior and posterior longitudinal ligaments and by discs of fibrocartilage, called intervertbral discs, positioned between opposing faces of adjacent vertebrae.

Each vertebrae includes an anterior body and a posterior arch. The posterior arch includes two pedicles and two laminae that join together to form the spinous process. A transverse process is laterally positioned at the transition from the pedicles to the laminae.

Both the spinous process and transverse process provide for attachment of fibrous tissue, including muscle. Two inferior articular processes extend downward from the junction of the laminae and the transverse process. Two superior articular processes extend upward from the junction.

The articular processes of adjacent vertebrae form the facet joints. The inferior articular process of one vertebra articulates with the superior articular process of the vertebra below.

Facet joints allow for movement of the spine in all directions. The facet joints are gliding joints because the articular surfaces glide over each other. Arthritis, degenerative disc disease and other various degenerative conditions can result in the need to fuse the facet joints together.

Facet joint fusion can reduce or eliminate pain and/or complications experienced by patients with degenerating facet joints. Facet fusion often involves destruction of the facet by decorticating the opposing articulating surfaces and packing bone growth promoting substances such as grafts or synthetic materials into the space between the articular processes.

The facet joints are generally small as compared to the intervertebral space. Consequently, limited amounts of bone-growth promoting substances may be inserted into the joint. Some of the bone-growth promoting substances tend to disperse post-operatively resulting in a less robust fusion. Furthermore, the overlying fibrous tissue may further disperse the bone-growth promoting substances as a result of contact, friction, and/or the in-growth of fibrous mass. These and other factors may result in pseudoarthrosis or inadequate fusion.

Many techniques have been developed to treat conditions associated with the facet joint. These techniques are generally classified either as resurfacing the facet joint or fusing the facet joint.

The facet resurfacing techniques at least partially cover at least one of the superior facet and the inferior facet. An example of one such facet resurfacing implant is disclosed in Soboleski et al., U.S. Pat. No. 7,371,238. In certain conditions, at least one of the implants is placed on one side of the spine such as to treat scoliosis.

Dooris et al., U.S. Pat. No. 7,101,398, discloses a prosthetic facet joint ligament that includes first and second components that are attached to the superior facet and the inferior facet. A flexible material interconnects the first and second components to allow movement of the facet joint.

Blain, U.S. Patent Publication No. 2005/0177240, describes a facet prosthesis that includes an implant that is placed between the superior facet and the inferior facet. An elongated retaining member extends through the implant, the superior facet and the inferior facet to retain the implant in the facet joint.

The facet fusion techniques seek to treat facet joint problems by preventing a superior facet from moving with respect to an inferior facet. One configuration includes a cylindrical implant with a threaded outer surface. The implant is screwed into a hole that is drilled along a joint line between the superior facet and the interior facet. Two such examples of these types of devices are described in Petersen, U.S. Pat. No. 7,708,761, and Pavlov et al., U.S. Patent Publication No. 2006/0064099.

Farris, U.S. Patent Publication No. 2007/0135814, describes an implant that includes a central leg that is positioned between the superior facet and the inferior facet. An upper leg of the implant is attached to the superior facet and a lower leg of the implant is attached to the inferior facet.

Kraus et al., U.S. Patent Publication No. 2006/0190081, discloses several different techniques for stabilizing a facet joint. One technique places a band around the superior facet and the inferior facet to prevent them from moving with respect to each other.

### SUMMARY OF THE INVENTION

An embodiment of the invention is directed to a facet fusion implant that includes a first drill guide and a second drill guide. The first drill guide includes a first main body portion and an alignment tab. The first main body portion has a first aperture and a second aperture formed therein. The alignment tab extends from the first main body portion. The second drill guide includes a second main body portion, a first extension and a second extension. The second main body portion has a third aperture formed therein. The first extension and the second extension extend from the second main body portion.

Another embodiment of the invention is directed to a facet fusion implant that includes a first gripping region, a second gripping region, a third gripping region and a bone growth region. The first gripping region, the second gripping region and the third gripping region each have a plurality of teeth formed thereon.

The bone growth region is located between at least one of the first gripping region, the second gripping region and the third gripping region. A thickness of the bone growth region is less than a thickness of the first gripping region.

Another embodiment of the invention is directed to a facet fusion system that includes a first drill guide, a second drill guide and a facet fusion implant. The first drill guide has a first aperture formed therein. The second drill guide has a second aperture formed therein. A position of the first aperture on the first drill guide is offset from a position of the second aperture on the second drill guide.

The facet fusion implant has at least one bone growth region and at least one gripping region. The at least one gripping region has a thickness that is larger than a diameter of the first aperture and the second aperture.

Another embodiment of the invention is directed to a method of fusing a joint. A bony region is provided that has a first bony surface and a second bony surface that are positioned in an adjacent relationship so that a gap is defined therebetween.

A first drill guide is positioned proximate the gap. The first drill guide has a first drill guide aperture formed therein. A first drill bit is extended through the first drill guide aperture. The first drill bit is rotated to cause a first aperture to be formed. The first aperture partially extends into at least one of the first bony surface and the second bony surface. The first drill guide is removed from the gap.

A second drill guide is positioned proximate the gap. The second drill guide has a second drill guide aperture formed therein. A second drill bit is extended through the second drill guide aperture. The second drill bit is rotated to cause a second aperture to be formed. The second aperture partially extends into at least one of the first bony surface and the second bony surface and wherein the first aperture is offset from the second aperture.

A fusion implant is urged into the gap. The fusion implant includes a first gripping region, a second gripping region and a first bone growth region. The first gripping region extends into the first aperture and the second gripping region extends into the second aperture to prevent the first bony surface from moving with respect to the second bony surface.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 is a perspective view of a first drill guide for use with the facet fusion system.

FIG. 2 is a top view of the first drill guide.

FIG. 3 is a side view of the first drill guide.

FIG. 4 is an end view of the first drill guide.

FIG. 5 is a perspective view of a second drill guide for use with the facet fusion system.

FIG. 6 is a top view of the second drill guide.

FIG. 7 is a side view of the second drill guide.

FIG. 8 is an end view of the second drill guide.

FIG. 9 is a perspective view of a facet fusion implant for use with the facet fusion system.

FIG. 10 is a top view of the facet fusion implant.

FIG. 11 is a sectional view of the facet fusion implant taken along a line 11-11 in FIG. 10.

FIG. 12 is a sectional view of the facet fusion implant taken along a line 12-12 in FIG. 10.

FIG. 13 is a photograph of the first drill guide being used in conjunction with a facet fusion process.

FIG. 14 is a photograph of the second drill guide being used in conjunction with the facet fusion process.

FIG. 15 is a photograph of apertures formed using the first drill guide and the second drill guide.

FIG. 16 is a photograph of the facet fusion implant inserted proximate the apertures.

### DETAILED DESCRIPTION OF THE PREFERRED EMBODIMENTS

An embodiment of the invention is to a facet fusion system as illustrated in the figures. The facet fusion system includes a component for preparing the facet joint. Then a facet implant is inserted into the prepared region.

The facet joint is prepared using a first drill guide 20 and a second drill guide 22. As described in more detail below, the first drill guide 20 and the second drill guide 22 are used in series to prepare a region in a facet joint for receipt of an implant.

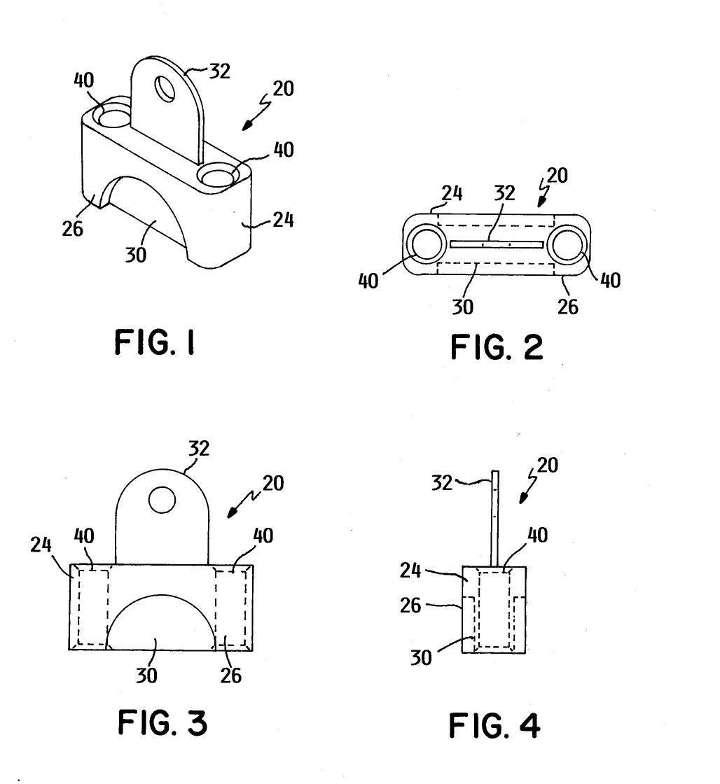

The first drill guide 20 includes a main body portion 24, as illustrated in FIGS. 1-4. The main body portion 24 may have a generally rectangular shape. A size of the main body portion 24 should be sufficiently large to facilitate holding and positioning by a person using the first drill guide 20. However, the main body portion 24 should be relatively small to facilitate placement adjacent the facet joint where the implant is to be inserted.

The first main body portion 24 may have a width up to about 1 inch and, in certain embodiments, may be between about 0.50 and 0.70 inches. The first main body portion 24 may have a height of up to about 1 inch and, in certain embodiments, may be between about 0.20 and 0.50 inches. The first main body portion 24 may have a thickness of up to about 1 inch and, in certain embodiments, may be between 0.10 and 0.30 inches.

At least one of the side surfaces 26 of the first main body portion 24 may include a recess 30. The recess 30 defines a location for a person using the first drill guide 20 to position his/her fingers when holding the first drill guide 20.

The recess 30 may have a width that is approximately the same as a width of a person's finger who is using the first drill guide 20 to facilitate a person using the first drill guide 20 to accurately position the person's finger on the first drill guide 20 without looking at the first drill guide 20. In certain embodiments, the recess 30 has a width of up to about 1 inch.

A front edge of the recess 30 may be curved. The curvature of the front edge may generally conform to a curvature of the person's finger. Using the curved front edge further enhances the ability of the person to accurately position the person's finger on the first drill guide without looking at the first drill guide 20.

The first main body portion 24 has at least one aperture 40 formed therein. In certain embodiments, the first main body portion 24 includes two apertures 40 that are positioned adjacent to opposite edges of the first main body portion 24.

The aperture 40 extends between upper and lower ends of the first main body portion 24, as most clearly illustrated in FIGS. 3 and 4. In certain embodiments, the at least one aperture 40 has a diameter of less than about 0.25 inches. In other embodiments, the at least one aperture 40 has a diameter of between about 0.05 and 0.15 inches. The aperture 40 is formed with a diameter that is approximately the same as a diameter of a drill bit that is intended to be used with the first drill guide 20.

The drill bit may have a diameter that is greater than a spacing between the bones in the facet joint such that when the first drill guide 20 is positioned adjacent the facet joint so that the tab 32 extends into the space between the bones in the facet joint such that the diameter of the drill bit is greater than the space between the bones in the facet joint. In certain embodiments, the drill bit may have a diameter of about 3/32 of an inch.

Using this configuration causes the drill bit to remove a portion of the bones when the drill bit is extended through the at least one aperture 40 and into the bones. Removing a portion of the bones prepares the bones for bone growth around the implant, which plays an important role in successfully fusing the facet joint. More details on the fusion process are set forth below.

A tab 32 extends from an end of the first main body portion 24. The tab 32 may be positioned approximately intermediate the apertures 40, as illustrated in FIGS. 1-4. The tab 32 facilitates locating a space between the bones in the facet joint. The tab 32 may have a thickness that is considerably less than a thickness of the first main body portion 24.

The tab 32 may have a width of up to about 0.50 inches and, in certain embodiment, is between about 0.20 and 0.40 inches. The tab 32 may have a height of up to about 0.50 inches and, in certain embodiment, is between about 0.20 and 0.40 inches. The tab 32 may have a thickness of up to about 0.10 inches and, in certain embodiment, is between about 0.01 and 0.05 inches.

While it is possible for the tab 32 and the first main body portion 24 to be fabricated from a single piece of material, it is also possible to fabricate the tab 32 separately from the first main body portion 24 and then attach the tab 32 to the first main body portion 24. A variety of techniques may be used to attach the tab 32 to the first main body portion 24 depending on the materials from which the tab 32 and the first main body portion 24 are formed.

In certain embodiments, the tab 32 may be resiliently deformable to enhance the ability to guide the tab 32 between the bones in the facet joint. Once the tab 32 is no longer subjected to the force causing the deformation, the tab 32 may return to the initial configuration.

The tab 32 is illustrated as being oriented substantially parallel to the first main body portion 24. Such a configuration enables the drill bit to be guided between the bones of the facet joint. It is also possible to orient the tab 32 at an angle with respect to the first main body portion 24. Such a configuration could be used to facilitate drilling more deeply into one of the bones of the facet joint.

The first drill guide 20 may be fabricated from a variety of materials using the concepts of the invention. The material used to fabricate the first drill guide 20 may be sufficiently rigid to resist deformation during use. The material used to fabricate the first drill guide 20 may also facilitate sterilization. In certain embodiments, the first drill guide 20 is fabricated from a metallic material that meets the preceding characteristics.

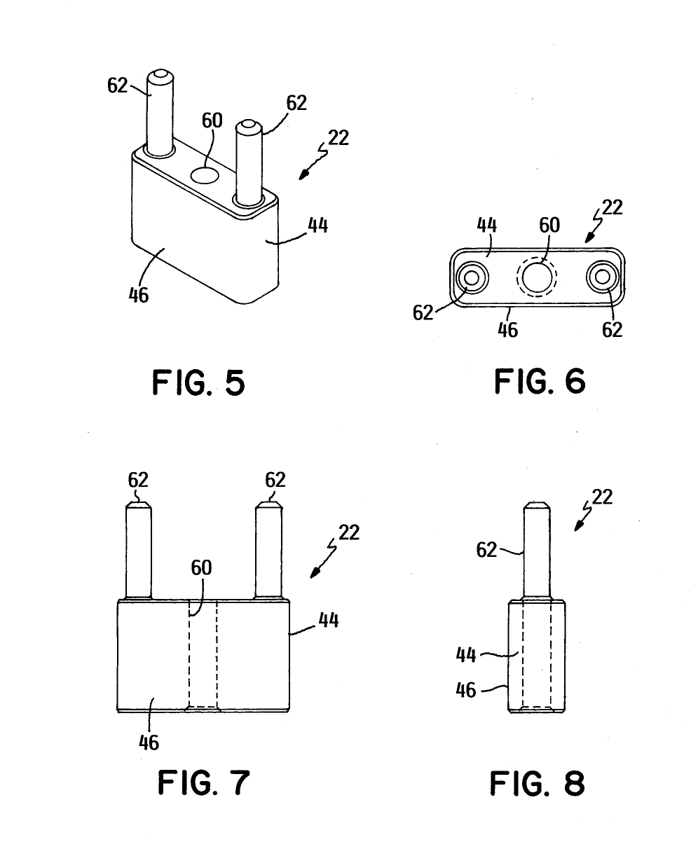

The second drill guide 22 includes a second main body portion 44, as illustrated in FIGS. 5-8. The second main body portion 44 may have a generally rectangular shape. A size of the second main body portion 44 should be sufficiently large to facilitate holding and positioning by a person using the second drill guide 22. However, the second main body portion 44 should be relatively small to facilitate placement adjacent the facet joint where the implant is to be inserted.

The second main body portion 44 may have a width up to about 1 inch and, in certain embodiments, may be between about 0.50 and 0.70 inches. The second main body portion 44 may have a height of up to about 1 inch and, in certain embodiments, may be between about 0.20 and 0.50 inches. The second main body portion 44 may have a thickness of up to about 1 inch and, in certain embodiments, may be between 0.10 and 0.30 inches.

Similar to the first main body portion 24, at least one of the side surfaces 46 of the second main body portion 44 may include a recess (not shown). The recess defines a location for a person using the second drill guide 22 to position his/her fingers when holding the second drill guide 22.

The second main body portion 24 has at least one aperture 60 formed therein. The aperture 60 may be positioned at an intermediate location on the second main body portion 44. The aperture 60 extends between upper and lower ends of the second main body portion 44, as most clearly illustrated in FIG. 7.

In certain embodiments, the at least one aperture 60 has a diameter of less than about 0.25 inches. In other embodiments, the at least one aperture 60 has a diameter of between about 0.05 and 0.15 inches.

The aperture 60 is formed with a diameter that is approximately the same as a diameter of a drill bit that is intended to be used with the second drill guide 22. Similar to the drill bit used in conjunction with the first drill guide 20, the drill bit used in conjunction with the second drill guide 22 may have a diameter of about 3/32 of an inch.

Using this configuration causes the drill bit to remove a portion of the bones when the drill bit is extended through the at least one aperture 40 and into the bones. Removing a portion of the bones prepares the bones for bone growth around the implant, which plays an important role in successfully fusing the facet joint. More details on the fusion process are set forth below.

At least one extension 62 extends from an end of the second main body portion 44. The at least one extension 62 may be formed with a cylindrical shape. In certain embodiments, the at least one extension 62 has a diameter that is slightly smaller than a diameter of the drill bit used in conjunction with the first drill guide 20. The at least one extension 62 may have a diameter of up to about 0.25 inches and, in certain embodiments, is between about 0.05 and 0.15 inches. Using this configuration enables the extension to extend into the hole in the facet joint formed by the drill bit.

The at least one extension 62 may be mounted in a spaced-apart configuration. The mounting of the at least one extension 62 may conform to a spacing between the apertures 40 on the first drill guide 20. Using such a configuration enables the extensions 62 to extend into holes formed by the drill bit using the first drill guide 20 to thereby retain the second drill guide 22 in a stationary position with respect to the facet joint while the second drill guide 22 is used.

While it is possible for the at least one extension 62 and the second main body portion 44 to be fabricated from a single piece of material, it is also possible to fabricate the at least one extension 62 separately from the second main body portion 44 and then attach the at least one extension 62 to the second main body portion 44. A variety of techniques may be used to attach the at least one extension 62 to the second main body portion 44 depending on the materials from which the at least one extension 62 and the second main body portion 44 are formed.

The at least one extension 62 is illustrated as being oriented substantially parallel to the second main body portion 44. Such a configuration enables the drill bit to be guided between the bones of the facet joint. It is also possible to orient the at least one extension 62 at an angle with respect to the second main body portion 44. Such a configuration could be used to facilitate drilling more deeply into one of the bones of the facet joint.

The second drill guide 22 may be fabricated from a variety of materials using the concepts of the invention. The material used to fabricate the second drill guide 22 may be sufficiently rigid to resist deformation during use. The material used to fabricate the second drill guide 22 may also facilitate sterilization. In certain embodiments, the second drill guide 22 is fabricated from a metallic material that meets the preceding characteristics.

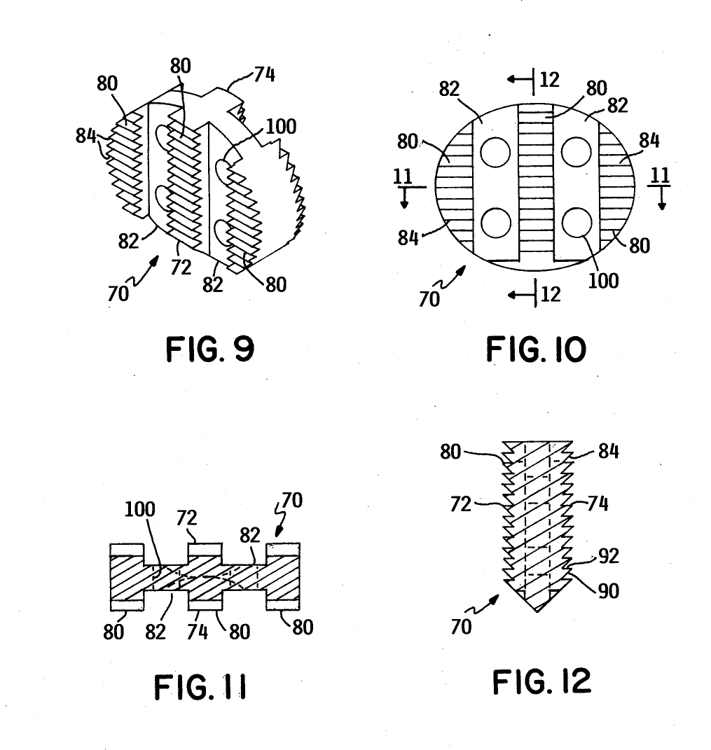

Another aspect of the invention relates to a facet implant 70, which is illustrated in FIGS. 9-12. The facet implant 70 is inserted into the opening formed in the facet joint using the first drill guide 20 and the second drill guide 22.

In certain embodiments, the facet implant 70 may have a generally cylindrical configuration, as illustrated in the figures. While the facet implant 70 may be formed with other shapes, forming the facet implant 70 with the cylindrical configuration minimizes the potential of corners of the facet implant 70 engaging portions of the person's body that are adjacent to the facet joint where the facet implant 70 is used and thereby causing damage to such areas.

The facet implant 70 generally includes a first side face 72 and a second side face 74, which are generally oriented opposite each other. In certain embodiments, the first side face 72 and the second side face 74 may be oriented substantially perpendicular to each other.

The first side face 72 and the second side face 74 may be shaped substantially similar to each other. As such, only the shape of the first side face 72 will be discussed herein. The first side face 72 may include at least one gripping region 80 and at least one bone growth region 82.

The at least one gripping region 80 may have a texture that reduces the potential of the facet implant 70 moving after being inserted into the facet joint. Moving of the facet implant 70 in the time period shortly after being inserted into the facet joint is important so that bone may grow around and/or through the facet implant 70. This bone growth may limit long-term movement of the facet implant 70 in the facet joint.

The gripping region 80 may have a plurality of teeth 84 extending from the surface thereof. In certain embodiments, the teeth 84 may substantially cover an outer surface of the gripping region 80.

The teeth 84 may be mounted in a spaced-apart configuration. The spacing between the teeth 84 may be substantially equal. In certain embodiments, there may be between about 10 and 30 teeth 84 across a surface of the gripping region 80. Forming the gripping region with teeth 84 in the preceding range provides a balance of ease of insertion and the ability to retain the facet implant 70 in the facet joint.

As illustrated in FIG. 12, the teeth 84 may each be defined by a leading surface 90 and a trailing surface 92. The leading surface 90 may be oriented at an angle of between about 30° and about 90° with respect to a horizontal plane. In certain embodiments, the leading surface 90 may be oriented at an angle of 45° with respect to a horizontal plane. Orienting the leading surface 90 in this configuration enhances the ability to insert the facet implant 70 into the facet joint.

The trailing surface 92 may be oriented at an angle that is greater than the angle at which the leading surface 90 is oriented. In certain embodiments, the trailing surface 92 may be oriented at an angle of about 90° with respect to a horizontal plane. Orienting the trailing surface 92 in this configuration enhances the ability of the facet implant 70 to remain in the facet joint after insertion.

The gripping regions 80 may be provided in three locations on the first side face 72. Each of the locations may generally conform to a location at which the hole is formed in the facet joint, as is discussed in more detail below.

Intermediate each of the gripping regions 80 is one of the bone growth region 82. The bone growth regions 82 may have a height that is less than a height of the gripping regions 80. The bone growth regions 82 may be configured to be oriented adjacent to the portions of the facet joint that are between the holes formed in the facet joint.

The bone growth regions 82 may have a substantially flat surface that enables the bone growth region 82 to slide over the surfaces of the facet joint during the insertion process. The bone growth regions 82 may have at least one aperture 100 formed therein. The aperture 100 may extend substantially between the first side face 72 and the second side face 74. The aperture 100 thereby facilitates growth of bone through the facet implant 70. Growth of the bone thereby reduces the potential of the facet implant moving after the insertion process.

The number and size of the apertures 100 may be selected to balance the strength of the facet implant 70 against the amount of bone growth. In certain embodiments, there are two apertures. The total area of the apertures is up to about 30 percent of the surface area of the bone growth region 82.

To facilitate insertion of the facet implant 70 into the facet joint, a leading edge of the facet implant 70 may be tapered to a point. In certain embodiments, the tapered sides may be oriented at an angle of between about 30° and about 60°. In other embodiments, the sides are tapered at an angle of about 45°. While the tapered sides are illustrated as being symmetrical, it is possible for the tapered sides to be oriented in different configurations.

Since the facet joint involves relatively small bones that are positioned in a relatively close relationship, one of the important aspects of successfully performing a facet fusion is to accurately identify a joint line between the components of the facet joint.

Tissue overlying the facet joint in which it is desired for the facet implant to be inserted is moved to expose facet joint. A probe or other similar surgical tool is then used to identify the joint line.

Next, the surface of the facet joint may be prepared using a rasp. The rasp may be taped into the joint several times and then removed. The process may be repeated after the rasp is rotated about 180°.

The first drill guide 20 is positioned adjacent to the facet joint 104 so that the tab 32 at least partially extends into the facet joint 104. The tab 32 thereby enhances the ability to accurately position the first drill guide 20 with respect to the facet joint 104.

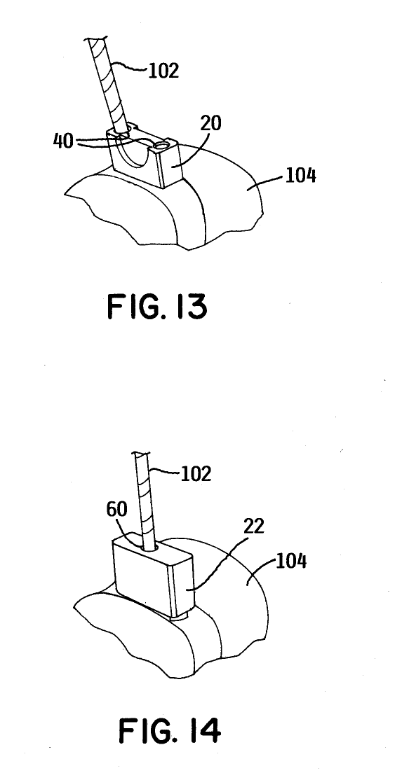

Next, a drill bit 102 is inserted into each of the apertures 40, as illustrated in FIG. 13. As is discussed above, the drill bit 102 may have a diameter of about 3/32 of an inch. The drill bit 102 is then extended into the facet joint 104 to a depth that is slightly larger than the size of the facet implant 70. The drill bit 102 should not be extended too far into the facet joint 104 because the drill bit 102 may damage tissue on a back side of the facet joint 104. After the drilling is completed, the first drill guide 20 is removed.

The second drill guide 22 is positioned adjacent the facet joint 104 so that the extensions 62 extend into the apertures in the facet joint 104 that were formed using the drill bit 102 in conjunction with the first drill guide 20. The drill bit 102 is inserted into the aperture 60, as illustrated in FIG. 14.

Similar to the drill bit 102 used in conjunction with the first drill guide 20, the drill bit 102 used in conjunction with the second drill guide 22 may have a diameter of about 3/32 of an inch. In other embodiments, the drill bit 102 used in conjunction with the first drill guide 20 may have a diameter that is different than the drill bit 102 used in conjunction with the second drill guide 22.

The drill bit 102 is then extended into the facet joint 104 to a depth that is slightly larger than the size of the facet implant 70. The drill bit 102 should not be extended too far into the facet joint 104 because the drill bit may damage tissue on a back side of the facet joint 104. After the drilling is completed, the second drill guide 22 is removed.

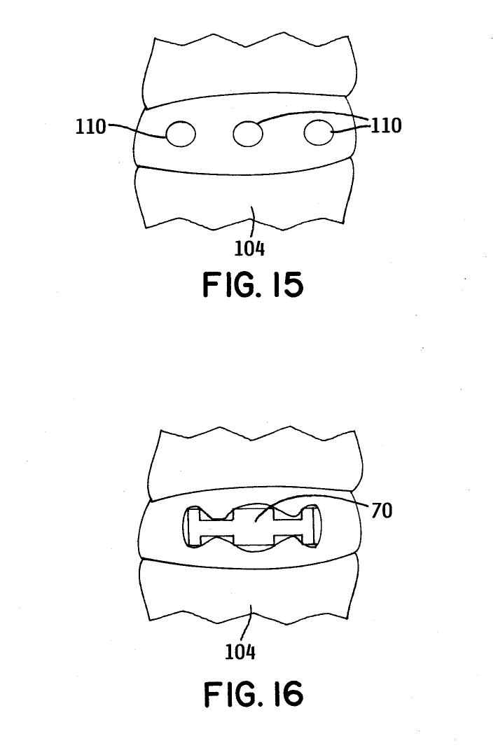

The facet joint 104 thereby has three facet joint apertures 110 formed therein, as illustrated in FIG. 15. It is possible to increase the size of the facet joint apertures 110 by overdrilling using a larger drill bit. In certain embodiments, the overdrilling is done with a drill bit (not shown) having a diameter that is about 7/64 of an inch.

The facet implant 70 may be grasped manually or using a curved forceps as part of the process of inserting the facet implant 70 into the prepared region in the facet joint 104. Each of the gripping regions 80 is aligned with one of the facet joint apertures 110.

Because it is desired for the facet implant 70 to fit snugly in the facet joint 104, it may be necessary to tap the facet implant 70 into place. After the facet implant 70 has been inserted, it may be substantially recessed in the facet joint 104, as illustrated in FIG. 16.

In the preceding detailed description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The preceding detailed description, therefore, is not to be taken in a limiting sense, and the scope of the present invention is defined by the appended claims.

It is contemplated that features disclosed in this , as well as those described in the above applications incorporated by reference, can be mixed and matched to suit particular circumstances. Various other modifications and changes will be apparent to those of ordinary skill.

1. A facet fusion system comprising:
a first drill guide comprising:
a first main body portion having a first aperture and a second aperture formed therein; and
an alignment tab extending from the first main body portion; and
a second drill guide comprising:
a second main body portion having a third aperture formed therein;
a first extension that extends from the second main body portion; and
a second extension that extends from the second main body portion.

2. The facet fusion system of claim 1, wherein the first extension has a diameter that is no larger than a diameter of the first aperture and wherein the second extension has a diameter that is no larger than a diameter of the second aperture.

3. The facet fusion system of claim 1, wherein a position of the third aperture on the second main body portion is offset from a position of the first aperture and the second aperture on the first main body portion.

4. The facet fusion system of claim 1, wherein the alignment tab is intermediate the first aperture and the second aperture.

5. The facet fusion system of claim 1, wherein the third aperture is intermediate the first extension and the second extension.

6. The facet fusion system of claim 1, and further comprising an implant comprising a first gripping region, a second gripping region and a third gripping region, wherein a spacing between the first gripping region and the second gripping region is approximately the same as a spacing between the first aperture and the second aperture.

7. The facet fusion system of claim 6, wherein the first gripping region, the second gripping region and the third gripping region each comprise a plurality of teeth.

8. The facet fusion system of claim 6, wherein the implant comprises:
a first bone growth region intermediate the first gripping region and the second gripping region; and
a second bone growth region between the second gripping region and the third gripping region.

9. The facet fusion system of claim 8, wherein the first bone growth region and the second bone growth region both have a height that is less than the height of the first gripping region.

10. The facet fusion system of claim 8, wherein the first bone growth region and the second body growth region each have at least one aperture formed therein that extends through the implant.

11. The facet fusion system of claim 6, wherein the implant comprises a first major surface and a second major surface that is opposite the first major surface, wherein the first gripping region, the second gripping region and the third gripping region each extend from the first major surface and the second major surface.

12. The facet fusion system of claim 6, wherein the implant has a profile that is circular or oval.

13. A facet fusion implant comprising:
a first gripping region, a second gripping region and a third gripping region that each have a plurality of teeth formed thereon; and
a bone growth region located between at least one of the first gripping region, the second gripping region and the third gripping region, wherein a thickness of the bone growth region is less than a thickness of the first gripping region.

14. The facet fusion implant of claim 13, wherein the facet fusion implant has a first side surface and a second side surface, wherein the gripping region is formed on at least one of the first side and on the second side and wherein the bone growth region is formed on at least one of the first side and the second side.

15. The facet fusion implant of claim 13, wherein the facet fusion implant has a profile that is circular or oval.

16. The facet fusion implant of claim 13, wherein the facet fusion implant comprises a proximal edge and a distal edge, wherein the plurality of teeth are oriented to facilitate insertion of the facet fusion implant moving toward the distal edge and wherein the plurality of teeth are oriented to resist movement of the facet fusion implant toward the proximal edge.

17. The facet fusion implant of claim 13, wherein the facet fusion implant comprises a proximal edge and a distal edge and wherein at least a portion of the distal edge is tapered.

18. The facet fusion implant of claim 13, wherein the facet fusion implant has at least one aperture formed therein and wherein the at least one aperture is located in the bone growth region.

19. A facet fusion system comprising:
a first drill guide having a first aperture formed therein;
a second drill guide having a second aperture formed therein, wherein a position of the first aperture on the first drill guide is offset from a position of the second aperture on the second drill guide; and
a facet fusion implant having at least one bone growth region and at least one gripping region, wherein the at least one gripping region has a thickness that is larger than a diameter of the first aperture and the second aperture.

20. The facet fusion system of claim 19, wherein the second drill guide comprises a main body portion and at least one extension attached to the main body portion.

21. The facet fusion system of claim 19, wherein the first drill guide further comprises an alignment tab that extends from a surface thereof, wherein the alignment tab has a diameter that is not larger than a diameter of the first aperture.

22. The facet fusion system of claim 19, wherein the facet fusion implant has a first side surface and a second side surface, wherein the gripping region is formed on at least one of the first side and on the second side and wherein the bone growth region is formed on at least one of the first side and the second side.

23. The facet fusion system of claim 19, wherein the gripping region has a plurality of teeth formed thereon.

24. The facet fusion system of claim 19, wherein the facet fusion implant comprises a plurality of the gripping regions and a plurality of the bone growth regions and wherein the gripping regions and the bone growth regions are oriented in an alternating configuration.

25. The facet fusion system of claim 19, wherein a thickness of the bone growth region is less than a thickness of the gripping region.

26. The facet fusion system of claim 19, wherein the facet fusion implant has a circular or oval profile.

27. The facet fusion system of claim 19, wherein the facet fusion implant has at least one aperture formed therein and wherein the at least one aperture is located in the bone growth region.

28. A method of fusing a joint comprising:
providing a bony region having a first bony surface and a second bony surface that are positioned in an adjacent relationship so that a gap is defined therebetween;
positioning a first drill guide proximate the gap, wherein the first drill guide has a first drill guide aperture formed therein;
extending a first drill bit through the first drill guide aperture;
rotating the first drill bit to cause a first aperture to be formed, wherein the first aperture partially extends into at least one of the first bony surface and the second bony surface;
removing the first drill guide from the gap;
positioning a second drill guide proximate the gap, wherein the second drill guide has a second drill guide aperture formed therein;
extending a second drill bit through the second drill guide aperture;
rotating the second drill bit to cause a second aperture to be formed, wherein the second aperture partially extends into at least one of the first bony surface and the second bony surface and wherein the first aperture is offset from the second aperture; and
urging a fusion implant into the gap, wherein the fusion implant comprises a first gripping region, a second gripping region and a first bone growth region, wherein the first gripping region extends into the first aperture and the second gripping region extends into the second aperture to prevent the first bony surface from moving with respect to the second bony surface.

29. The method of claim 28, wherein the first drill bit guide comprises a first main body portion and an alignment tab that extends from the first main body portion and wherein the method further comprises extending the alignment tab into the gap.

30. The method of claim 28, wherein the second drill bit guide comprises a second main body portion and a first extension and wherein the method further comprises extending the first extension into the first aperture.

31. The method of claim 28, wherein the first bone growth region is located between the first gripping region and the second gripping region.

32. The method of claim 28, and further comprising:
providing a second drill guide aperture in the first drill guide;
extending the first drill bit through the second drill guide aperture;
rotating the first drill bit to cause a third aperture to be formed, wherein the second aperture partially extends into at least one of the first bony surface and the second bony surface; and
urging the fusion implant into the gap, wherein the fusion implant further comprises a third gripping region that extends into the third joint aperture to prevent the first bony surface from moving with respect to the second bony surface.

33. The method of claim 33, wherein the first joint aperture, the second joint aperture and the third joint aperture are generally aligned in a linear orientation.

34. The method of claim 33, wherein the second joint aperture is between the first joint aperture and the third joint aperture.

35. The method of claim 28, wherein the fusion implant has a first side and a second side, which is located opposite the first side, wherein at least a portion of the first gripping region is formed on the first side and wherein at least a portion of the first gripping region is formed on the second side.

36. The method of claim 28, wherein the fusion implant has at least one aperture formed therein and wherein the method further comprises growing bone through the at least one aperture.

37. The method of claim 28, wherein the first gripping region has a plurality of teeth formed thereon and wherein the plurality of teeth engage at least one of the first bony surface and the second bony surface to impede movement of the fusion implant with respect to at least one of the first bony surface and the second bony surface.

38. The method of claim 28, wherein the bony region is a facet joint.
